(19) United States
(12) Patent Application Publication
(10) Pub. No.: US 2014/0296164 A1
(43) Pub. Date: Oct. 2, 2014

(54) Compositions and methods of use for cell targeted inhibitors of the Cystic Fibrosis transmembrane regulator associated ligand

(71) Applicants: Andrew Peter Mallon, Lincoln, RI (US); Alvin C. Bach, II, Stonington, CT (US)

(72) Inventors: Andrew Peter Mallon, Lincoln, RI (US); Alvin C. Bach, II, Stonington, CT (US)

(73) Assignee: CALISTA THERAPEUTICS, INC., Lincoln, RI (US)

(21) Appl. No.: 14/229,851

(22) Filed: Mar. 29, 2014

Related U.S. Application Data
(60) Provisional application No. 61/806,753, filed on Mar. 29, 2013.

(51) Int. Cl. C07K 7/06; A61K 45/06; A61K 38/08

(52) U.S. Cl. CPC C07K 7/06; A61K 38/08; A61K 45/06
USPC 514/21.6; 530/323

(57) ABSTRACT

The present invention describes peptide drugs that inhibit the interaction between CAL and CFTR, and other proteins in cystic fibrosis and other diseases. These invented drugs have been chemically optimized to impart solubility, stability, cell permeability, mucus penetration, intracellular targeting and sequestration, increased potency and non-immunogenicity, while conserving and imparting efficacy. This renders these compositions suitable for human use, which is exemplified by use in the treatment of cystic fibrosis.

Provisional application No. 61/806,753, filed Mar. 29, 2013

This invention was made with US government support under grant numbers 1R43NS074651-01, 1R43HL120469-01 and 1R43DK101304-01 awarded by the National Institutes of Health. The government has certain rights in the invention.

### FIELD OF THE INVENTION

The present disclosure relates to stable, soluble, cell permeant and targeted PDZ domain inhibitor peptide drugs, and more particularly to inhibitors of the cystic fibrosis transmembrane regulator (CFTR) associated ligand (CAL). The invention further relates to pharmaceutical compositions comprising said compounds, and methods for the preparation and use of said pharmaceutical compositions.

### BACKGROUND ART

Prior art according to US20120071396 are referred to for comparison.

US20120071396 contains a general discussion of the possibility of making certain changes to prior art sequences and such possible modifications. Such changes include the addition of a Cell Permeability Peptide (CPP) to impart cell uptake, using unnatural D amino acids and/or D-retro-inverso conformations to improve stability, and addition of sequence linkages. However, making these changes do not provide predictable and discrete changes and interfere with efficacy.

Thus, there remained a need to invent a novel peptide that contains innovative improvements that provide cell permeability and stability without loss of efficacy.

### SUMMARY OF THE INVENTION

According to one aspect of the present invention there is provided a CAL inhibitor (iCAL) peptide, selected from the group consisting of: X₁-YGRKKRRQRRR-X₂-WQTRV-X₃ and X₁-YGRKKRRQRRR-X₂-ANSRWPTSII-X₃, or a variant thereof wherein X₁ are selected from the group consisting of NH₂ and acetyl, X₂ are a cleavable linker, including glycoyl, or a spacer and X₃ are selected from the group consisting of OH and an amide.

In an embodiment X₁ is alkyl so as form an ester at the C-terminus. In an embodiment alkyl is a C1-6 alkyl. In a further embodiment the C-terminus is acetyl.

In an embodiment the X₂ linker contains a cleavable bond or a spacer. In an embodiment the linker may be cleaved by hydrolysis, including by intracellular esterases. In an embodiment the linker comprises an ester linkage. In an embodiment the linker comprises a group-O—(CH₂)ₙ—COO— wherein n is an integer from 1 to 6.

The amino acids may be in the L-configuration or the D-configuration, or in the D-conformation in a retro inverso sequence. In an exemplified embodiment they are L-amino acids.

The present inventors were able to provide cell permeability using a N-terminus addended TAT (YGRKKRRQRRR) sequence but required linking it to the WQVTRV or ANSRWPTSII iCAL sequence by a linker such as a glycoyl group (glycoyl=HO—CH2-COOH). Without being bound by any theory the introduction of a linker serves several purposes. In one instance, it reduces steric hindrance of WQVTRV, allowing it to bind, having been distanced from the TAT sequence appreciably to minimize both its ability to interact with the binding sequence and WQVTRV and the target. In addition, if the linker contains a cleavable bond that can be readily hydrolyzed by intracellular esterases, in one particular embodiment, the WQVTRV or active sequence is released inside the cell to act with no hindrance. This has the additional benefit in a preferred embodiment of effectively targeting or sequestering the sequence in the first cells it encounters and enters. In one highly preferred embodiment this results in an inventive composition, formulation, and method of use that preferentially distributes a topical dose of the invention to the pulmonary epithelial cells and prevents and/or lessens the subsequent active transport out of the epithelial cell into the systemic circulation, where it can be transported to distant sites, where toxicity and safety issue may arise. We define topical as more broadly meaning the application to the apical side of the epithelial or outermost environment facing aspect of any cell of a tissue. In addition, this invention decreases clearance of the invention, and provides for a local potentiation of the dosage effect which allows for much lower (including >50 times lower) concentrations to be used to achieve a therapeutic effect (10 μM in this invention-CT007, vs 500 μM WQVTRV and other prior sequences with BIOPORTER). (CT007, Acety-YGRKKRRQRRR-glycoyl-WQVTRV-amide).

In an embodiment the present inventors also extended the sequence with an additional amide group on the N-terminus and an acetyl group on the C-terminus that provides improved stability; and binding efficacy by more faithfully presenting the peptide sequence as it would be found and bind endogenously.

In another preferred embodiment of this invention, we describe a method of use wherein the drug is administered to a Cystic Fibrosis (CF) patient with any CF mutation.

In this invention, we have discovered a sequence that retains activity when it comprises inherent cell permeability from a CPP sequence that would otherwise inhibit its efficacy. This provides an invention that is both drug like and safe, limiting systemic distribution and potential toxicity through an innovative drug delivery mechanism that is precisely targeted at a topical delivery formulation inherent in the composition and the method of its use.

Importantly, the changes we invented to the peptides are not predictable in their effect on cell permeability, stability and activity or efficacy of the peptides. Thus, one skilled in the art would know that significant experimentation would be needed to identify the unobvious changes that provide for cell penetration while retaining bioeffectiveness. The invention described herein teaches a specific method of use and composition of matter that was precisely designed and discovered to create a drug-like composition that conserves and greatly enhances efficacy. Further, certain of the changes described in the prior art actually resulted in activity loss and were thus not obvious improvements that could result in a predictable outcome by one skilled in the art.

This invention describes compositions and methods for stabilizing the cell membrane location of the CFTR, a chloride ion channel that is membrane-unstable in cystic fibrosis, and implicated in other diseases.

In certain embodiments, the invention imparts cell permeability, solubility, targeted intracellular release, enhanced in vivo stability, non-immunogenicity and physiochemical properties that allow formulation into a dosage form, including an inhaled dry powder or solution, or any dosage form that allows for topical dosing and immediate uptake in epithelial or other surface cell types.

In certain embodiments, the invention is a method of treating or preventing cystic fibrosis, the method comprising administering to a subject in need an effective amount of the invention or a pharmaceutically acceptable salt thereof.

In certain embodiments, the step of administering the invention includes the compound in a pharmaceutically acceptable composition.

In certain embodiments, the subject is a human, a companion animal, or a feed animal.

In certain embodiments, the effective amount of the invention is in a range 0.0001 to about 100 mg per kg of body weight per day.

In certain embodiments, the method further comprises the step of monitoring the subject to determine invention efficacy and tolerance, and adjust treatment levels accordingly.

In certain embodiments, the invention is administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, and by inhalation, injection, infusion, continuous infusion, localized perfusion bathing target cells directly, via a catheter, via lavage, in creams, in lipid compositions.

In certain embodiments, additional therapeutic agent(s) are administered to the subject at the same time or within the time period for bioeffectiveness of the invention, including: PDZ modulating agents, CFTR modulators, correctors and/or potentiators, mucolytics, anti-infective agents, corticosteroids and/or bronchodilators.

In certain embodiments, we describe a method wherein iCAL inhibitors require combination with therapeutic agents that improve CFTR membrane expression in order to enable iCAL inhibitors to provide a therapeutic effect.

In certain embodiments the described compositions are useful in modulating the localization, activation and interaction of cellular proteins, including the ABC transporters, including the CFTR protein, as a treatment to prevent, lessen the severity of or cure a disease in a patient comprising administering to said patient one of the compositions as defined herein, and said disease is selected from CFTR mediated disease that should include cystic fibrosis, asthma, smoke induced chronic obstructive pulmonary disease (COPD), chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear plasy, Pick's disease, several polyglutamine neurological disorders such as Huntington's, spinocerebullar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, dry-mouth diseases or Sjogren's disease, Osteoporosis, Osteopenia, bone healing and bone growth (including bone repair, bone regeneration, reducing bone resorption and increasing bone deposition), Gorham's Syndrome, chloride channelopathies such as myotonia congenita (Thomson and Becker forms), Bartter's syndrome type III, Dent's disease, hyperekplexia, epilepsy, hyperekplexia, lysosomal storage disease, and Primary Ciliary Dyskinesia (PCD), a term for inherited disorders of the structure and/or function of cilia, including PCD with situs inversus (also known as Kartagener syndrome), PCD without situs inversus and ciliary aplasia. In a specific embodiment this invention describes a method of treatment of diarrhea, including secretory diarrhea, muscositis, and radiation and chemically induced CFTR-mediated disease, with more specifically, a method of treating epithelia at specific locations or throughout the gastrointestinal tract.

In a certain embodiment, this invention has a method of use as a chemical, biological or radiological countermeasure by providing decorporation or a protection, mitigation or treatment.

In another embodiment, this invention has particular efficacy in diarrhea, including secretory diarrhea.

In another embodiment, this invention treats dry eye and dry mouth.

Other features and advantages of the invention will be obvious from the claims and detailed description.

Objectives, features and advantages of the embodiments shall become apparent as the description thereof proceeds when considered in connection with the accompanying illustrative drawings.

### DETAILED DESCRIPTION OF THE INVENTION

CF is the single most prevalent life-shortening genetic disorder among white Caucasian individuals worldwide (Boat (1997) NIH Consensus Development Conference on Genetic Testing for Cystic Fibrosis. Bethesda, Md.: National Institutes of Health;). CF is a recessive genetic disease that impairs the ability of epithelial cells to transport chloride ions, resulting in an abnormally viscous mucus secretion, most notably in the lungs, pancreas, liver, intestines, sinuses, and sex organs. This results in an average life expectancy of 37.4 years for the ˜30,000 CF patents in the US (2009 Cystic Fibrosis Foundation). CF is most common among European Caucasians (1 in 25 are carriers). Despite the low numbers of patients, CF is the most widespread life-limiting inherited disease in this population with 70% of patients diagnosed by 2 years of age. There is a high annual cost for CF patient care (Lieu, et al. (1999) Pediatrics 103:e72) and effective treatment remains a major unmet medical need.

Current treatments largely consist of symptom and co-morbidity management rather than direct disease modification, including preemptory and aggressive control of respiratory infections, decrease in mucous levels (physical therapy, mucolytics), pancreatic enzyme supplementation, good nutrition/life-style, vitamin supplementation, saline inhalations, etc (Rowe, et al. (2005) New England Journal of Medicine 352:1992-2001). Antibiotics are constantly required because CF patients develop chronic antibiotic-resistant infections. DNA gene therapy to restore CFTR has so far failed, primarily because of inefficient gene delivery strategies and cDNA recombination issues (Tate, et al. (2005) Expert Opinion on Drug Delivery 2:269-280).

In patients, as pulmonary function declines, mechanical breathing becomes necessary, including invasive and non-invasive modes (Moran, et al. (2009) Cochrane Database of Systematic Reviews). Critically, once predicted-for-age lung function declines to ˜20-30%, a lung or heart-lung transplant is the only remaining treatment (Scott, et at (1988) The Lancet 332:192-194, Orenstein, et al. (1991) Chest 100:1016-1018), and this remains substantially unsuccessful. Thus, a patient's health can quickly deteriorate past a point at which they are no longer viable surgical candidates (Belkin, et al. (2006) American Journal of Respiratory and Critical Care Medicine 173:659-666). With the transplant waiting period as long as two years (Kerem, et al. (1992) New England Journal of Medicine 326:1187-1191), the majority of CF patients (90%) die in early adulthood (MacLusky, et al. (1990) Disorders of the respiratory tract in children 5th ed. 692-729). CF therefore remains a major unmet medical need. Herein we target a new therapeutic paradigm by developing a first-in-class, chronic use peptide CFTR stabilizer drug for CF that can be a standalone therapy or one used in conjunction with existing therapies.

CF is caused by various genetic defects in the CFTR gene at the q31.2 locus of chromosome 7 (Rowe, et al. (2005) New England Journal of Medicine 352:1992-2001, Riordan, et al. (1989) Science 245:1066-1073). The CFTR gene encodes a chloride channel that resides in the apical membrane of epithelial cells, where it maintains the proper ionic balance in fluid secretions. While there are many different types of CFTR mutations, the most common is a deletion (delta) of a phenylalanine (F) codon at position 508 (deltaF508), involved in ˜90% of all CF cases (Vinay Kumar, at al. (2007)). With deltaF508-CFTR, the CFTR protein does not fold properly in the endoplasmic reticulum (ER)Cheng, et al. (1990) Cell 63:827-834, Kerem (2004) Current Opinion in Pulmonary Medicine 10:547-552) causing decreased ER-Golgi trafficking and increased ER degradation and any inserted channel to gate chloride ion flux in a severely impaired manner (Dalemans, et al. (1991) Nature 354:526-528). Additionally, instead of being properly trafficked and maintained at the apical membrane, the protein is prematurely degraded in the lysosome (Cholon, et al. (2010) American Journal of Physiology-Lung Cellular and Molecular Physiology 298:L304-L314). While recent efforts have tried to identify compounds that can affect the gating and ER trafficking deficiencies (‘potentiators’ and ‘correctors’, respectively), any CFTR that does express on the membrane surface is highly unstable, limiting their therapeutic value. Thus, stabilization of deltaF508-CFTR is an attractive and substantially validated therapeutic goal in CF and other diseases.

In addition to the deltaF508 mutation specifically, there are many other mutations that are progressively rare, including the G551D. The described invention can be used as a therapy in all types of CF (Heim, et al. (2001) Genet Med 3:168-176).

Membrane stability of CFTR is regulated by a series of PDZ-class proteins. PDZ (post-synaptic density 95, Discs large, ZO-1) domains are special binding zones on proteins that mediate their interactions with other proteins. PDZ domains are specialized protein-interaction motifs that are frequently found in multi-domain scaffolding proteins and are known to be involved in the assembly of large molecular complexes at defined locations in the cell. PDZ scaffolds are also integral in the dynamic trafficking of synaptic proteins by assembling cargo complexes for transport by molecular motors such as dynein and kinesin along microtubules (Kim, et al. (2004) Nat Rev Neurosci 5:771-781). As key coordinators that direct synaptic protein composition and structure, PDZ scaffolds are themselves highly regulated by synthesis and degradation, cellular allocation and post-translational alteration, and are a well-validated therapeutic target. PDZ domains are critical for membrane expression of CFTR (Swiatecka-Urban, et al. (2002) Journal of Biological Chemistry 277:40099-40105).

One PDZ protein is the CFTR-associated ligand (CAL), which, without being bound by any particular theory, decreases CFTR localization at the membrane surface by directing it to lysosomes for degradation (Cholon, et al. (2010) American Journal of Physiology-Lung Cellular and Molecular Physiology 298:L304-L314). However, CFTR also non-selectively interacts with several Na⁺/H⁺ exchanger regulatory factors (NHERF), most notably NHERF1 and NHERF2, which counteract CAL by stabilizing CFTR in the membrane.

Recently, CAL inhibitor (iCAL) peptide sequences have been identified. These selectively inhibit CAL, but not NHERF, increasing deltaF508-CFTR at the membrane resulting in increased chloride conductance from CF patient-derived bronchial epithelium (Cushing, et al. (2010) Angewandte Chemie International Edition 49:9907-9911, Roberts, et al. (2012) PLoS Comput Biol 8:e1002477). These ‘iCAL’ peptides are competitive inhibitors selectively binding to the CAL PDZ domain, preventing interaction with CFTR (Cushing, et al. (2010) Angewandte Chemie International Edition 49:9907-9911).

However, these isolated sequences are unsuitable as drug compounds and required substantial medicinal chemistry optimization and improvement. These iCAL sequences were: 1) not inherently cell permeable, and thus unable to reach their intracellular CAL target and have a pharmacological effect. This made them unusable as drugs as demonstrated in FIG. 1, CT008, which is WQVTRV alone and CT002 which is WQVTRV with BIOPORTER reagent. Neither had any effect on CFTR compared to baseline. 2) Insoluble, due to a high proportion of hydrophobic amino acids in the sequences. This results in a critical loss of efficacy because to act as a drug a compound must dissolve in aqueous fluids to have any effect. 3) Low efficacy, requiring 500 μM concentrations for effects. 500 μM is a very high dosage that indicates very low efficacy despite Ki binding efficacies for the sequences at ˜1 μM. The 500 μM concentration actually required is an indication of the low drug-like potential of the sequences. In addition, 500 μM was only achieved with BIOPORTER reagent, which has no place or alternative in clinical or in vivo use. 4) Low stability, this is as a result of an easily digested linear L amino acid sequence without any protections to prevent or delay degradation, and 5) not specifically tissue targeted, which is a composition and method of use we have invented to allow for safe administration to the pulmonary and bronchial epithelium in a highly preferred embodiment of this invention, and with appropriate formulation development can be used for delivery to other epithelial body surfaces.

Our invention of optimized iCAL drug compounds described here represents a breakthrough for selective stabilization of deltaF508-CFTR as a therapy option in CF and other diseases by maintaining functional CFTR at the cell surface. Using medicinal peptide chemistry, we have now designed, synthesized and tested an optimized exemplary lead ‘CT007’.

Current disease modifying treatments most similar to CT007 and the inventions described herein are Vertex's CFTR modulators: VX-770, VX-809 and VX-661 (Clancy, et al. (2011) Thorax 67:12-18, Van Goor, et al. (2009) Proceedings of the National Academy of Sciences 106:18825-18830, Van Goor, et al. (2011) Proceedings of the National Academy of Sciences 108:18843-18848). These drugs share the same therapeutic aim as these inventions, but use a different mechanism of action for directly facilitating and restoring physiological function to the CFTR.

In CF patients, deltaF508-CFTR exhibits three functional defects in the way CFTR is processed. 1. CFTR is folded improperly, resulting in increased ER degradation and decreased Golgi trafficking. 2. CFTR at the apical membrane has a reduced open probability (Po), resulting in reduced ion flow; and 3. CFTR is removed from the apical membrane and degraded in lysosomes at a higher frequency than normal due to pathologically strong interactions with CAL. We target the latter of these therapeutic targets with a CFTR stabilizer iCAL drug.

We describe clinical additivity or even synergy for combination therapy for other drugs with these inventions described herein because preliminary tests with VX-809, a CFTR corrector, and CT007 proved successful (see FIGS. 1, 2 and 3). Stabilization of CFTR at the membrane surface is predicted to have the greatest potential for therapeutic efficacy to treat the thick mucus secretions that are the hallmark of CF pathology (Rowe, et al. (2005) New England Journal of Medicine 352:1992-2001) and potentially for other diseases. Our hypothesis is that targeting CAL with these inventions will have the greatest near-term therapeutic potential due to the stabilization of CFTR resulting in longer membrane CFTR half-life and increased CFTR ion flow.

Until now, there was no success at developing a stabilizer compound that would allow deltaF508-CFTR to remain in the membrane for extended periods of time. Such an action provides chloride conduction that inhibits thick CF mucus secretions. The failure was due, at least in part, to the difficulties associated with the overlapping specificities of the PDZ domains on (1) CAL, the negative regulator, and (2) the NHERF family, the positive regulator of CFTR function. It has been difficult to develop a compound or biologic that would target CAL without disturbing the beneficial NHERF-deltaF508-CFTR interactions. Non-drug-like in vitro sequences that block the interaction and binding of CFTR and CAL by competitive displacement are described in the prior art (Cushing, et al. (2010) Angewandte Chemie International Edition 49:9907-9911, Wolde, et al. (2007) Journal of Biological Chemistry 282:8099-8109).

Early peptide CAL inhibitors (iCAL), include iCAL36 and kCAL01 (See prior art). However, these inhibitors did not have the inherent cell permeability, cell targeting or solubility, required to reach the intracellular CAL target nor enhanced stability. The preliminary research into the prior art was undertaken using 500 μM iCAL sequences with BioPORTER®, a lipid based reagent system that can provide in vitro but not in vivo cell permeability. We have changed and improved this peptide series and invented CT007, an inherently stable and cell permeable clinical lead with potency, efficacy, epithelial cell targeting, and cell uptake at low concentrations (10 μM).

6. Rationally designed inventive peptide lead compounds, described herein, have enhanced stability, potency, bioavailability, intracellular targeting and efficacy. We designed, synthesized and tested a series of peptides incorporating moieties that impart inherent intracellular delivery properties with D-amino acids and retro-inverso conformations to provide stability. We are specialists in peptide drug design, delivery, research, and development. We invented novel drug-like sequences based on iCAL36 and kCal01 sequences (Cushing, et al. (2010) Angewandte Chemie International Edition 49:9907-9911, Wolde, et al. (2007) Journal of Biological Chemistry 282:8099-8109, Iskandarsyah (2008) Chem Biol Drug Des. 72:27-33, Chan, et al. (2000) Oxford University Press Fmoc Solid Phase peptide synthesis, A Practical Approach) and the directed changes included: 1) the substitution of D-amino acids to generate the chiral D form of the natural L form, known to impart stability on peptide drugs (Milton, et al. (1992) Science 256:1445-1448, Schumacher, et al. (1996) Science 271:1854-1857). In addition, D peptides induce less of an immunogenic reaction (Welch, et al. (2007) Proceedings of the National Academy of Sciences 104:16828-16833). Selective enhancement of peptide stability is advantageous, to decrease proteolytic enzyme digestion (Adessi, et al. (2002) Current Medicinal Chemistry 9:963-978). 2) D-Retro-Inverso conformation describes an analogue with D-amino acids in the reversed sequence (Guichard, et al. (1994) Proceedings of the National Academy of Sciences 91:9765-9769, Cardo-Vila, et al. (2010) Proceedings of the National Academy of Sciences). This works best on small peptides that do not rely upon a persistent secondary protein structure for target binding. D-retro-inverso versions of CPPs are still effective with enhanced cell penetration and an added benefit of being protease resistant (Mason (2010) Future Medicinal Chemistry 2:1813-22). Reduced proteolysis also increases intracellular peptide concentration (Brugidou (1995) Biochem. Biophys. Res. Commun. 214:685-693). 3) Incorporation of selected CPP moiety confers the ability for active transport into cells, where pharmacological effects are needed. This was accomplished by the prior art by use of the in vitro BioPorter® lipid reagent, which is not for human use (Cushing, et al. (2010) Angewandte Chemie International Edition 49:9907-9911). We have utilized and tested several CPPs and here we used the TAT transcription factor (Wadia, et al. (2002) Current Opinion in Biotechnology 13:52-56). This provides efficacy and enhances our freedom to operate. This Tat sequence has been shown safe and effective in humans (NA-1: NCT00728182, XG-102: NCT01570205 (Aarts, et al. (2002) Science 298:846-50, Borsello, et al. (2003) Nat Med 9:1180-1186)). 4) Incorporation of a cleavable glycoyl linker to the CPP. This allowed release of the active moiety by intracellular esterases without significant loss in overall stability or the generation of toxic moieties. 5) Amide capping of C-terminus, and Acetyl capping of N-terminus: this provides added stability, by reducing degradation, and increases binding efficacy by replicating better the binding domain conformation being in sequence than C terminus. 6) Cyclic peptide conformations were developed to provide a highly stable structure to enhance CPP efficacy, facilitate uptake and advanced well as clinical leads (e.g. cyclosporine, DDAVP) (Cunningham, et al. (2009) The Open Enzyme inhibition Journal, Cascales, et al. (2011) Journal of Biological Chemistry 286:36932-36943, Lattig-Tunnemann, et al. (2011) Nat Commun 2:453, Mimetogen (2011) Mimetogen Pharmaceuticals Inc.). Proteins need to be unraveled and undergo a ‘lock and key’ type interaction with an enzyme before being available for digestion. This ‘stapling’ effect of peptides prevents digestion whilst maintaining pharmacological activity (Mason (2010) Future Medicinal Chemistry 2:1813-22). The cyclic peptides are also designed with an optimized CT007 sequence incorporating different length glycine bridges to form cyclo-octa, cyclo-deca, and cyclo-dodeca peptides. Glycine without the sidechain found in other amino acids was chosen to further minimize non-specific interactions. Cyclic peptides are resistant to proteolysis because they lack free termini. These cyclic peptides also restrict the conformational space each cyclic peptide can sample compared to the linear peptide. D-amino acid CPPs are used for stability.

Peptidomimetic compositions can be made up of any mixture of non-natural structural parts, which are typically from at least three structural groups: residue linkage groups other than the natural amide bond (“peptide bond”) linkages; non-natural residues in place of naturally occurring amino acid residues; residues that induce secondary structural mimicry, i.e., induce or stabilize a secondary structure, e.g., a beta turn, gamma turn, beta sheet, alpha helix conformation, and the like; or other changes that confer resistance to proteolysis. For example, a polypeptide can be characterized as a mimetic when one or more of the residues are joined by chemical means other than an amide bond.

Individual peptidomimetic segments can be linked by amide bonds, non-natural and non-amide chemical bonds other chemical bonds or coupling means including, for example, glutaraldehyde, N-hydroxysuccinimide esters, glycoyl, bifunctional maleimides, N,N-dicyclohexylcarbodiimide (DCC) or N,N-diisopropyl-carbodiimide (DIC). Linking groups alternative to the amide bond and glycoyl bond include, for example, ketomethylene (e.g., —C(═O)—CH2- for —C(═O—NH—), aminomethylene (CH2-NH), ethylene, olefin (CH—CH), ether (CH2-O), thioether (CH2-S), tetrazole (CN4-), thiazole, retroamide, thioamide, or ester(Jung-Mo Ahn, et al. (2002) Mini Reviews in Medicinal Chemistry 2:463-73).

A peptidomimetic embodiment can be characterized as being made up of one or more non-natural residues in place of naturally occurring amino acid residues. Non-natural residues are identified in the art. Particular non-limiting examples of non-natural residues useful as mimetics of natural amino acid residues are mimetics of aromatic amino acids include, for example, but not limited to, D- or L-naphylalanine; D- or L-phenylglycine; D- or L-2 thieneylalanine; D- or L-1, -2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenyl-phenylalanine; and D- or L-2-indole(alkyl)alanines, where alkyl can be substituted or unsubstituted methyl, ethyl, butyl, pentyl, propyl, isopropyl, iso-butyl, hexyl, sec-isotyl, iso-pentyl, or a non-acidic amino acid. Aromatic rings of a non-natural amino acid that can be used in place a natural aromatic ring include, for example, but not limited to, thiazolyl, thiophenyl, pyrazolyl, benzimidazolyl, naphthyl, furanyl, pyrrolyl, and pyridyl aromatic rings.

Cyclic peptides or cyclized residue side chains can decrease propensity of a peptide to proteolytic degradation. Thus, certain embodiments embrace a peptidomimetic of the peptides disclosed and described herein, whereby one or more amino acid residue side chains are cyclized according to conventional methods. Also, back bone cyclization of the entire peptide is an embodiment, in some cases extension of the sequence is employed to conserve pharmacological efficacy

Mimetics of certain acidic amino acids can be generated by substitution with non-carboxylate amino acids while maintaining a negative charge; (phosphono) alanine; and sulfated threonine. Carboxyl side groups (e.g., aspartyl or glutamyl) can also be selectively modified by reaction with carbodiimides (R—N—C—N—R′) including, for example, but not limited to, 1-cyclohexyl-3(2-morpholinyl-(4-ethyl) carbodiimide or 1-ethyl-3(4-azonia-4,4-dimetholpentyl) carbodiimide. Aspartyl or glutamyl groups can also be converted to asparaginyl and glutaminyl groups by reaction with ammonium ions.

Lysine mimetics can also be made (and amino terminal residues can be altered) by reacting lysinyl with succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate.

Methionine mimetics can be generated by reaction with methionine sulfoxide. Proline mimetics of include, for example, but not limited to, pipecolic acid, thiazolidine carboxylic acid, dehydroproline, 3- or 4-methylproline, and 3,3,-dimethylproline.

Residues can be replaced by one or more amino acid (or peptidomimetic) residues of the opposite chirality. Any amino acid naturally occurring in the L-configuration can be replaced with the same amino acid or a mimetic, but of the opposite chirality, referred to as the D-amino acid.

As will be apparent to one skilled in the art, peptidomimetics of the present invention can also include one or more of the modifications described herein for derivatized peptides, e.g., a label, one or more post-translational modifications, or cell-penetrating sequence. While a peptide of this invention can be derivatized with by one of the above indicated modifications, it is understood that a peptide of this invention may contain more than one of the above described modifications within the same peptide.

To test for bioeffectiveness, for our exemplary invention to stabilize CFTR, we measured the chloride transport function homozygous deltaF508-CFTR cystic fibrosis human bronchial epithelial cells grown and differentiated on Snapwell™ filter inserts as CFTR agonist evoked short circuit (I_{SC}) current. The I_{SC} was the output of an Ussing epithelial voltage clamp apparatus after amiloride block of sodium current through the epithelial sodium channel (ENaC). ENaC current was monitored as the amiloride sensitive current component of the I_{SC}. The objective of this study was to measure the ability of various compounds, including but limited to CT002, CT008, CT003, CT004, CT005, CT006, CT007, and AT0011 (peptide control), to restore function to defective deltaF508-CFTR in CFhBE cell monolayers (epithelia) after a one day incubation period on the apical surface of the epithelia. See FIG. 4 for sequences and ID numbers.

These peptide compositions differed in their amino acid sequence to optimize solubility, intracellular permeability, cell targeting, stability and toxicity (CT003-CT007 shown in FIG. 1). The control peptides were non-optimized WQVTRV sequences (CT002 with BIOPORTER and CT008) and an off-target peptide control (AT0011).

CT003, CT004, CT005, CT006, CT007, and AT0011 were combined with the known corrector VX-809. CT002 (WQVTRV) was incubated with 10 μL of the cell permeability enhancing reagent BioPORTER™. Statistical comparisons were made using Dunnett's test and Student's t-test with significance evaluated at P<0.05. The I_{SC} current polarity convention used here records apical to basolateral sodium current and basolateral to apical chloride current as negative.

There is described herein particular structures of the exemplary embodiments. It will be obvious to those skilled in the art that several modifications and rearrangements of the parts can be made without departing from the spirit and scope of the underlying inventive concept and that the same is not limited to the particular forms herein shown and described except insofar as indicated by the scope of the appended claims.

### Indications for Use

A method of treatment to prevent, lessen the severity of or cure a disease in a patient comprising administering to said patient a peptide as claimed in claim 1, and said disease is selected from a disease that should include cystic fibrosis, asthma, smoke induced chronic obstructive pulmonary disease (COPD), chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear plasy, Pick's disease, several polyglutamine neurological disorders such as Huntington's, spinocerebullar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, dry-mouth diseases or Sjogren's disease, Osteoporosis, Osteopenia, bone healing and bone growth (including bone repair, bone regeneration, reducing bone resorption and increasing bone deposition), Gorham's Syndrome, chloride channelopathies such as myotonia congenita (Thomson and Becker forms), Bartter's syndrome type III, Dent's disease, hyperekplexia, epilepsy, hyperekplexia, lysosomal storage disease, and Primary Ciliary Dyskinesia (PCD), a term for inherited disorders of the structure and/or function of cilia, including PCD with situs inversus (also known as Kartagener syndrome), PCD without situs inversus and ciliary aplasia or a condition selected from the group consisting of diarrhea, dry eye and dry mouth diseases muscositis, and radiation and chemically induced CFTR-mediated disease.

In certain preferred embodiments, the present invention further comprises or is combined with another CFTR restorative therapy, including CFTR correctors, mis-sense correctors, CFTR potentiators, as well as mucolytics, bronchodilators, trafficking facilitators, such as PDZ1 and 2 modulatory drugs to induce a therapeutic effect in a patient such as improved respiration, decreased coughing and improved digestion.

In an embodiment, a patient diagnosed with CF can be administered with the inventive compounds in a concentration and using a mode of administration sufficient to retard CFTR degradation and improve CFTR function and enhance the efficacy of other treatments aimed at enhancing CFTR membrane expression and activity. This effect will result in improvement in the main pathological features of CF, including but not limited to improved respiration, and digestion.

Objectives, features and advantages of the embodiments shall become apparent as the description thereof proceeds when considered in connection with the accompanying illustrative drawings.

### BRIEF DESCRIPTION OF THE DRAWING FIGURES

The drawings provided illustrate several enabling disclosures of the present invention and its action.

FIG. 1 is a histogram demonstrating the comparative efficacy in human CF deltaF508 CFTR bronchial epithelium of several embodiments of the invention, compared to VX-809, a positive control that is a CFTR corrector and AT0011, a CPP peptide negative control.

FIG. 2 is a histogram displaying the significantly greater CFTR chloride current restoration induced by CT007 when added to VX-809 treatment that demonstrates the utility and additivity of CT007.

FIG. 3 is a graph of current vs time, showing the responsiveness of CFTR to antagonist and agonist applications. Images of data traces for each file were broken into images of I_{SC} data from incubations of particular compounds with images of both of the Vehicle and positive control incubated epithelia I_{SC} data in each of the images (parts 1 and 2) of a particular file (a and b). ENaC currents are not displayed but were measured and the values compared for statistical significance. Amiloride 30 μM is applied to block sodium channel currents, leaving only the CFTR channel current recorded under Ussing chamber voltage clamp. The application of agonists (forskolin, IBMX and Genistein) that stimulate CFTR activity. AT010 and VX-809 significantly enhances CFTR responses as shown by the greater magnitude of downward deflection in the current trace.

FIG. 4 shows CT007, a peptide containing WQVTRV, and CT009, a peptide containing ANSRWPTSII, two CAL binding sequences. Each has a cell permeable peptide (CPP) conferring moiety YGRKKRRQRRR added to it. This moiety renders the WQVTRV or ANSRWPTSII sequence inherently cell permeable to a level that allows therapeutic cellular delivery. In addition, WQVTRV is insoluble in water and thus unsuitable as a drug substance. The selection of the TAT moiety also provides sufficient hydrophilic groups to confer solubility is an additional improvement here. The C and N termini have been capped with acetyl and amide groups, respectively. This improvement was in order to enhance both 1) the stability of the peptide drug and 2) its efficacy. The binding sequence is derived from a naturally occurring peptide sequence that occurs in the middle of a peptide chain where the free COOH acid form would not be present. The incorporation of a glycoyl group between the two sequences was designed in order to 1) prevent steric hindrance of the TAT sequence upon the active binding sequence and 2) to impart tissue target specificity for topical delivery of the invention. In this embodiment, the glycoyl group is hydrolyzed by intracellular esterases. This serves to isolate and accumulate the drug substance within the bronchial epithelium. This improves efficacy, mitigates loss from rapid systemic clearance and reduces systemic toxicity by maintaining a local topical effect. As evidence of the improvement in efficacy, we note that WQVTRV and ANSRWPTSII are reliant on BIOPORTER®, which is a liposomal, not for human use, laboratory-only reagent, to provide cell delivery. Without BIOPORTER neither sequence functions. With BIOPORTER providing cellular delivery, WQVTRV and ANSRWPTSII only show efficacy at or above 500 μM concentration. At this concentration WQVTRV is largely insoluble and needs additional laboratory steps to solvate. These steps and BIOPORTER are not suitable for human use. Conversely, CT007 is highly effective at 10 uM and below, a fifty-fold improvement in reported efficacy, at least.

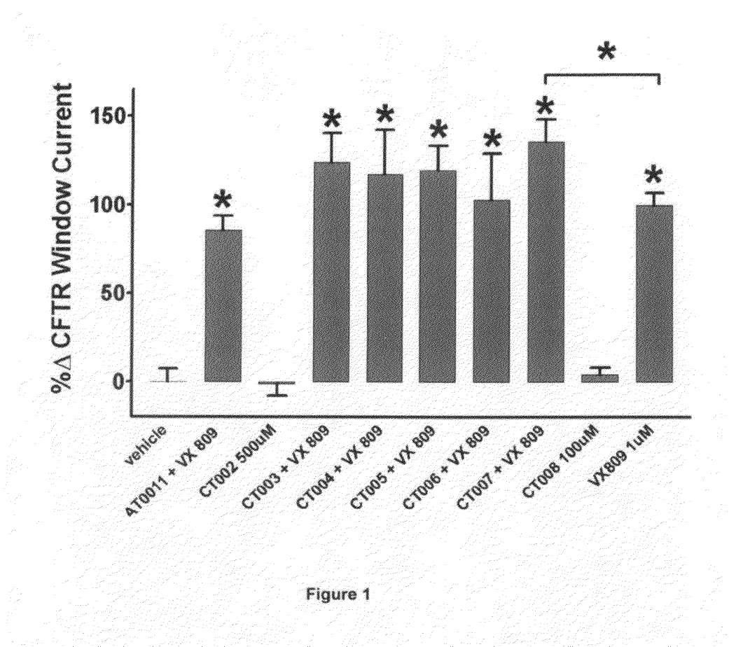

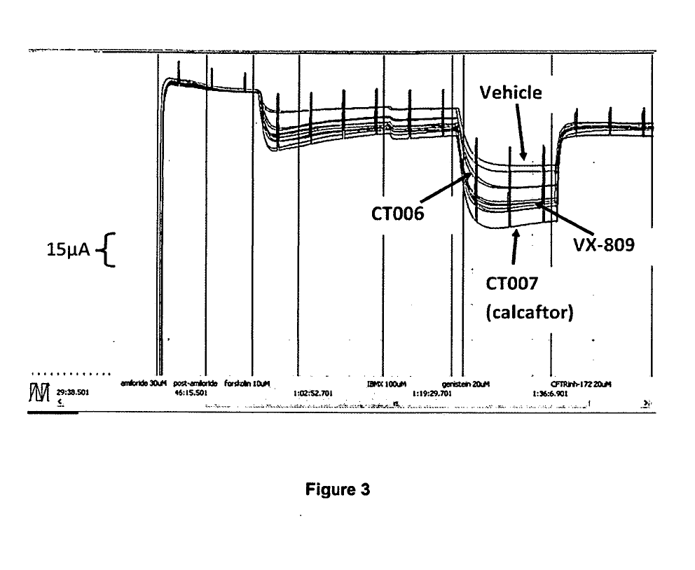

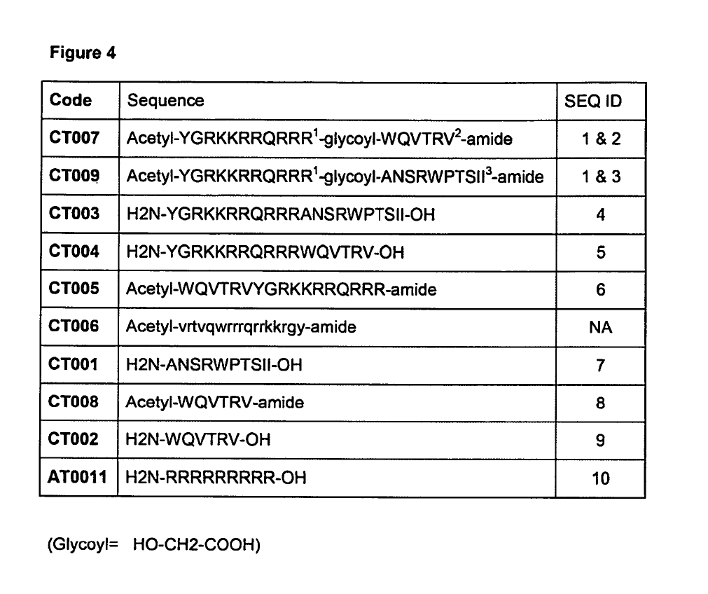

Referring now to the drawings, a first exemplary embodiment is illustrated and generally indicated in FIGS. 1, 2 and 3, which is the measurement of the chloride transport function of homozygous deltaF508-CFTR cystic fibrosis human bronchial epithelial cells grown and differentiated on Snapwell™ filter inserts as CFTR agonist evoked short circuit (I_{SC}) current. The I_{SC} was the output of an Ussing epithelial voltage clamp apparatus after amiloride block of sodium current through the epithelial sodium channel (ENaC). ENaC current was monitored as the amiloride sensitive current component of the I_{SC}. The objective of this study was to measure the ability of candidate Test Articles and controls (CT002 (WQVTRV+BIOPORTER), CT008 (WQVTRV alone), CT003, CT004, CT005, CT006, CT007, and AT0011 control) to restore function to defective deltaF508-CFTR in CFhBE cell monolayers (epithelia) after a one day incubation period on the apical surface of the epithelia. These peptide compounds differed in their amino acid sequence to optimize solubility, intracellular permeability, stability and toxicity (CT003-CT007 shown here). The control peptides were non-optimized sequences (CT002 and CT008) and an off-target peptide control (AT0011).

CT003, CT004, CT005, CT006, CT007, and AT0011 were combined with the known corrector VX-809 and CT002 was incubated with 10 μL of the cell permeability enhancing reagent BioPORTER™. Statistical comparisons were made using Dunnett's test and Student's t-test with significance evaluated at P<0.05. The I_{SC} current polarity convention used records apical to basolateral sodium current and basolateral to apical chloride current as negative.

All incubations were performed at 27±0.5° C. Test Articles were applied at their final concentrations to the apical surface of the cellular monolayers in 200 μL of Opti-MEM Reduced Serum Medium without addition of serum. Basolateral media, volume 2000 μL was changed but did not contain Test Articles. Test Article incubations were approximately 24 hours. 150 μL of cycloheximide (20 μg/mL) was applied to the apical surface of the epithelia for the final 2 hours of peptide treatment, total volume 350 μL. Cycloheximide prevented new protein synthesis that might be otherwise conflated with test article efficacy. This incubation protocol mimics the dilution and clearance effect that will be encountered by topical delivery to the lungs. Test articles are applied at t=0 h, 10 μM in 200 μL, to the apical surface. 2000 μL of basolateral medium provides an immediate dilution effect (test article: 0.91 μM), then at t=22 h, a further 350 μL of cycloheximide containing solution is further added, (test article: 0.85 μM). At t=24 hr, the test article containing solutions are removed and the bronchial epithelia are moved to test article free media.

Ussing Assay: All Ussing assays were performed at 27±0.5° C. and lasted for approximately 1 hour 40 minutes. The temperature was monitored throughout the experiment using a thermistor probe connected to the data acquisition system and inserted into the bath solution of one chamber. The bath temperature was adjusted manually to maintain the desired temperature. Test Articles were not maintained in the Ussing chamber solutions. Once the cellular monolayers were removed from the incubators just before the Ussing assay the apical solution was gently aspirated to prevent excess mixing with the 5 mL of apical bath solution in the Ussing chambers. Once mounted into Ussing chambers, epithelia were equilibrated for about 25 minutes in the chambers from the start of recording before application of voltage clamp and start of recording.

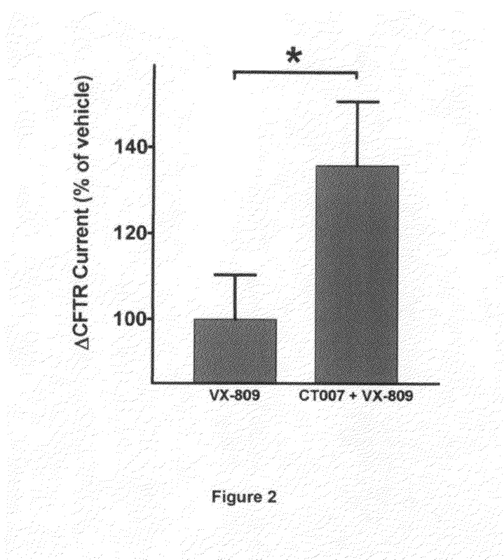

The overall conclusion from this work is that CT007, our inventive CAL inhibitor peptide drug, was confirmed to have 36% additive efficacy to VX-809, restoring defective deltaF508-CFTR and ENaC channel function in CF bronchial epithelium; refer to FIGS. 1 and 2 for a summary presentation of data. Of particular note in this data set is the low variability achieved with n=4 that resulted in a significant effect.

Importantly, Test Articles were applied apically, in a topical dose that was instantly subjected to dilution, such that the human CF epithelium was exposed to the effective concentration, only briefly. Also, prior to Ussing chamber testing, the test article solution was entirely removed. Despite this, CT007 treatment was still able to elicit effects with a long duration of action. This demonstrates a therapeutic drug action of at least 26 hours after transient topical apical dosing that should allow at least daily treatment and possibly longer.

Without being bound by any particular theory we posit that this invention sequesters the active sequence within the cell and/or the method of PDZ interaction with extant proteins causes long lasting changes that could include ubiquitination of the proteins resulting in their removal, thus requiring de novo protein synthesis to recover the prior conditions, including those that are pathological.

Results for I_{SC} measurements made on 9 Oct. 12 using patient code CFFT006F cells incubated for one day at 27° C. and assayed in symmetric solutions at 27° C.:

1. Epithelia treated with the positive control VX-809 1 μM and the Test Articles combined with VX-809 1 μM: AT0011 10 uM+VX-809 1 uM, CT003 10 uM+VX-809 1 uM, CT004 10 uM+VX-809 1 uM, CT005 10 uM+VX-809 1 uM, CT006 10 uM+VX-809 1 uM, and CT007 10 uM+VX-809 1 uM produced summed I_{SC} currents significantly above Vehicle treatment compared using Dunnett's test indicating robust increase of deltaF508-CFTR I_{SC}.
2. Summed I_{SC} currents from Test Article incubations alone were also compared with those generated by incubation with VX-809 1 μM. CT007 10 uM+VX-809 1 uM produced summed I_{SC} currents significantly above VX-809 1 μM treatment compared using Student's t-test.
3. Epithelia treated with VX-809 1 μM produced summed I_{SC} currents less than the expected level presumably because it was added as an apical incubation, but not in the basolateral media. The likely explanation for this is that VX-809 passed through the tight junctions between cells and was diluted by the basolateral media. Another possible explanation is that VX-809 has a lesser effect incubated on the apical side of CFhBE than on the basolateral side.
4. Epithelia treated with CT007 10 uM+VX-809 1 uM significantly increased the amiloride sensitive sodium current generated by ENaC compared to VX-809 alone using Student's test.
5. AT0011 10 uM+VX-809 1 uM significantly increased trans-epithelial resistance (TER) compared to Vehicle using Student's t-test.
6. CT007 10 uM+VX-809 1 uM significantly increased response to CFTRinh-172 compared with VX-809 1 uM compared using Student's t-test.
7. To optimize statistical comparisons, data from one epithelium incubated with CT004 10 uM+VX-809 1 uM and one epithelium incubated with CT006 10 uM+VX-809 1 uM were excluded to reduce the spread of data.

In another embodiment, a CF or other disease patient, who has any defect in CFTR protein is administered an effective dose of this invention. This inhibits the CAL-CFTR interaction and prevents the degradation or non-membrane sequestration and inactivation of CFTR.

### Comparative Example 1

We have designed and tested several analogues that incorporated changes that were posited in the prior art to provide obvious benefits, such as stability and inherent cell permeability that unexpectedly resulted in a loss and/or absence of efficacy. We define efficacy as the transduction of a significant pharmacological effect in the assay system described and presented in detail in the drawings. This lack of efficacy was also discovered to be inherent to the prior art sequences when used as described with BIOPORTER reagent to artificially provide in vitro cell permeability (Cushing, et at (2010) Angewandte Chemie International Edition 49:9907-9911).

The prior art sequences, in particular WQVTRV, are limited as therapeutic agents by inherent insolubility and a lack of cell permeability to allow targeting of the pharmacological receptor, which is crucial for bioeffectiveness and ultimately treatment of any PDZ-domain-dependent disease. This prior art specifically suggests in one embodiment the WrFKK sequence (Seq ID No 34) as a CPP. However, this CPP is particularly inefficacious for unknown reasons, and does not provide sufficient cell penetrating capacity to deliver the active sequence into the cell (Gomez, et al. (2011) Cell-Penetrating Peptides 683:465-471). This is an example where changes to the structure of the molecule do not predictably result in enhanced utility.

In addition, we have demonstrated that even using the prototypic CPP TAT sequence (YGRKKRRQRRR), which is established to provide excellent cell permeability, with the active sequence, this does not lead to conserved efficacy (FIGS. 1, 2 and 3, CT003, CT004 and CT005). The resultant composition may have cell permeability but it concomitantly loses pharmacological efficacy, which therein causes a loss in utility of the active sequence.

None of these changes that provide cell permeability were effective in retaining bioeffectiveness, and as shown in FIG. 1, these modified compounds did not provide conserved efficacy. This lack of an obvious benefit, demonstrates that the design of a ‘druggable’ derivative of an active sequence is not a predictable, especially since the ultimate aim is to retain sufficient bioeffectiveness to be able to treat a disease in the absence of toxicity; toxicity is inherently linked to high drug concentrations. Without being bound by any theory, we might predict that in some cases the steric hindrance provided by the CPP can interfere with the binding of the active sequence.

In addition, we found that the active sequence WQVTRV was insoluble at the effective concentrations for which it was intended to be used (FIG. 1, CT002, 500 μM) (Cushing, et al. (2010) Angewandte Chemie International Edition 49:9907-9911, Roberts, et al. (2012) PLoS Comput Biol 8:e1002477, Wolde, et al. (2007) Journal of Biological Chemistry 282:8099-8109, Madden (2011) DC0405US.P1:75, Vouilleme 2010, et al. (2010) Angewandte Chemie International Edition 49:9912-9916). To counter the propensity of hydrophobic amino acid residues, it was necessary to invent a sequence that comprised a balance of hydrophilic and hydrophobic residues that provided cell permeability and solubility at very low, yet efficacious, concentrations.

In another supposed obvious stability enhancing method in the prior art, the transformation of the active sequence into a D-retro inverso composition with a CPP sequence is suggested. However, as demonstrated by CT006 in FIG. 1, such an approach actually resulted in a loss of efficacy, despite the likely provision of cell permeability and a more stable composition.

1. A CAL inhibitor peptide, selected from the group consisting of: X₁-YGRKKRRQRRR-X₂-WQTRV-X₃ and X₁-YGRKKRRQRRR-X₂-ANSRWPTSII-X₃, wherein X₁ is an acetyl group; X₂ is a cleavable linker, including glycoyl, or a spacer; and X₃ is an amide group.

2. A method of targeted delivery to a cell, and more specifically, delivery and localized sequestration to the bronchial epithelium using a nebulizer solution or a dry powder inhalation administering a peptide according to claim 1 to a patient in need of such treatment.

3. A method of increasing CFTR protein abundance, trafficking, activation and residence time at the epithelial cell membrane administering a peptide to a patient in need of such treatment, according to claim 1.

4. A pharmaceutical composition comprising a peptide according to claim 1 and a pharmaceutically acceptable carrier.

5. A composition according to claim 4 adapted for administration by an administration route selected from a group including intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, and by inhalation, injection, infusion, continuous infusion, localized perfusion bathing target cells directly, via a catheter, via lavage, in creams, in lipid compositions.

6. A composition according to either one of claim 5, further comprising another CFTR restorative therapy, including CFTR correctors, mis-sense correctors, CFTR potentiators, and trafficking facilitators, such as PDZ1 and 2 NHERF1 modulators.

7. A method of treatment to prevent, lessen the severity of or cure a disease in a patient comprising administering to said patient a peptide as claimed in claim 1, and said disease is selected from a disease that should include cystic fibrosis, asthma, smoke induced chronic obstructive pulmonary disease (COPD), chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear plasy, Pick's disease, several polyglutamine neurological disorders such as Huntington's, spinocerebullar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, dry-mouth diseases or Sjogren's disease, Osteoporosis, Osteopenia, bone healing and bone growth (including bone repair, bone regeneration, reducing bone resorption and increasing bone deposition), Gorham's Syndrome, chloride channelopathies such as myotonia congenita (Thomson and Becker forms), Bartter's syndrome type III, Dent's disease, hyperekplexia, epilepsy, hyperekplexia, lysosomal storage disease, and Primary Ciliary Dyskinesia (PCD), a term for inherited disorders of the structure and/or function of cilia, including PCD with situs inversus (also known as Kartagener syndrome), PCD without situs inversus and ciliary aplasia or a condition selected from the group consisting of diarrhea, dry eye and dry mouth diseases muscositis, and radiation and chemically induced CFTR-mediated disease.

8. The method of use according to claim 3, wherein the CFTR protein mediated disease is cystic fibrosis, wherein the patient possesses one or more of the following mutations of human CFTR: deltaF508, G551D, R117H, G178R, G551S, G970R, G1244E, S1255P, G1349D, S549N, S549R, S1251N, E193K, F1052V G1069R, D110H, R347H, R352Q, E56K, P67L, L206W, A455E, D579G, S1235R, S945L, R1070W, F1074L, D110E, D1270N and D1152H on at least one allele and the method includes treating or lessening the severity of cystic fibrosis.

9. The method of use according to claim 5, wherein the pharmaceutical composition of the invention further comprises an additional agent selected from a mucolytic agent, a bronchodilator, an antibiotic, an anti-viral, an anti-invective agent, an anti-inflammatory agent, or a nutritional agent, or any combination thereof.


SEQUENCE LISTING (10 sequences)

SEQ ID NO: 1 - protein, 11 aa, artificial sequence
  Synthetic peptide
YGRKKRRQRRR

SEQ ID NO: 2 - protein, 6 aa, artificial sequence
  Synthetic peptide
WQVTRV

SEQ ID NO: 3 - protein, 10 aa, artificial sequence
  Synthetic peptide
ANSRWPTSII

SEQ ID NO: 4 - protein, 21 aa, artificial sequence
  Synthetic peptide
YGRKKRRQRRRANSRWPTSII

SEQ ID NO: 5 - protein, 17 aa, artificial sequence
  Synthetic peptide
WQVTRVYGRKKRRQRRR

SEQ ID NO: 6 - protein, 17 aa, artificial sequence
  Synthetic peptide
WQVTRVYGRKKRRQRRR

SEQ ID NO: 7 - protein, 10 aa, artificial sequence
  Synthetic peptide
ANSRWPTSII

SEQ ID NO: 8 - protein, 6 aa, artificial sequence
  Synthetic peptide
WQVTRV

SEQ ID NO: 9 - protein, 6 aa, artificial sequence
  Synthetic peptide
WQVTRV

SEQ ID NO: 10 - protein, 9 aa, artificial sequence
  Synthetic peptide
RRRRRRRRR